(19) United States
(12) Patent Application Publication
(10) Pub. No.: US 2002/0102678 A1
(43) Pub. Date: Aug. 1, 2002

(54) Staphylococcal gene, vgaC, conferring resistance to streptogramin A and related compounds

(76) Inventors: Nevine El Solh, Vincennes (FR); Julien Haroche, Paris (FR); Jeanine Allignet, Nanterre (FR)

Correspondence Address: FINNEGAN, HENDERSON, FARABOW,, GARRETT & DUNNER, L.L.P., 1300 I Street, N.W., Washington, DC, 20005, US

(21) Appl. No.: 09/817,764

(22) Filed: Mar. 27, 2001

Related U.S. Application Data
(60) Provisional application No. 60/197,372, filed on Apr. 14, 2000.

(51) Int. Cl. C12N 9/00; C07H 21/04; C12P 21/02; C12N 1/21; C12N 15/74

(52) U.S. Cl. 435/183; 435/252.3; 435/69.1; 435/320.1; 536/23.2

(57) ABSTRACT

A gene, which has been designated vgaC (1575 bp), encodes a putative ABC protein conferring resistance to streptogramin A and related antibiotics. The gene and fragments thereof, as well as the proteins and polypeptides they encode, are useful in diagnostic and therapeutic applications.

### CROSS-REFERENCE TO RELATED APPLICATIONS

This application is based on and claims the benefit of U.S. Provisional Application S. No. 60/197,372, filed Apr. 14, 2000 (attorney docket no. 03495.6046) The entire disclosure of this application is relied upon and incorporated by reference herein.

### BACKGROUND OF THE INVENTION

The present invention pertains to polynucleotides derived from a staphylococcal gene encoding resistance to streptogramin A and chemically related compounds. This invention also relates to the use of the polynucleotides as oligonucleotide primers or probes for detecting Staphylococcal strains that are resistant to streptogramin A and related compounds in a biological sample. The present invention is also directed to the full length coding sequence of the staphylococcal gene coding for resistance to streptogramin A from Staphylococcus and to the polypeptide expressed by these full length coding sequence. In addition, this invention relates to the use of the polypeptide to produce specific monoclonal or polyclonal antibodies that serve as detection means in order to characterize any staphylococcal strain carrying the gene encoding resistance to streptogramin A.

The present invention is also directed to diagnostic methods for detecting specific strains of Staphylococcus expected to be contained in a biological sample. The diagnostic methods use the oligonucleotide probes and primers as well as the antibodies of the invention.

Streptogramins (Sg) and related antibiotics are produced by streptomycetes and can be classified as A and B compounds, according to their basic primary structures (13). Compounds of the A group, including streptogramin A (SgA) and pristinamycin IIA (PIIA), are polyunsaturated cyclic macrolactones. Compounds of the B group, including streptogramin B (SgB) and pristinamycin IB (PIB), are cyclic peptide macrolactones. Compounds of groups A and B bind different targets in the peptidyltransferase domain of the 50S ribosomal subunit and inhibit protein elongation at different steps.

A-compounds provoke a conformational modification of the bacterial ribosome at the B compound binding site and decrease the dissociation constant of B compounds (14). Thus, A and B compounds, which are bacteriostatic when used separately, act synergistically, and when combined become bactericidal, mainly against Gram-positive bacteria. Natural mixtures, such as pristinamycin (Pt), synergistin, virginiamycin, and mikamycin, are used orally and topically. A semi-synthetic injectable Sg, Synercid®, consisting of a mixture of derivatives of streptogramins A and B (Dalfopristin and Quinupristin, respectively) is now available in hospitals for the treatment of infections due to Gram-positive cocci resistant to several other antibiotics (J. Antimicrob. Chemother., 1997, 40 (Suppl. A) and 1999, 44(Topic A), entire volumes).

Staphylococcal resistance to synergistic mixtures of A and B compounds (Pt MICs 2 mg.1⁻¹) is always associated with resistance to A compounds (PIIA MICs 8 mg.1¹), but not necessarily with resistance to B compounds (1). To date, seven genes encoding resistance to A compounds have been isolated from staphylococcal and enterococcal plasmids. Genes vat (8), vatB (2), vatC (5), satA (27), and satG (19, 32) encode related proteins (50.4-60.1% identical aa) conferring resistance to SgA and similar compounds. The proteins Vat and VatB exhibit acetyltransferase activity inactivating these antibiotics. The degenerate primers M and N were designed to detect any of these genes by PCR experiments (1). The staphylococcal vga (6) and vgaB genes (3) encode related ATP-binding proteins (58.8% identical aa), probably involved in the active efflux of A compounds (16).

There exists a need in the art for the identification of new genes that encode proteins conferring resistance to A compounds.

### SUMMARY OF THE INVENTION

This invention aids in fulfilling this need in the art. More particularly, cloning, sequencing, and distribution of a new staphylococcal gene, vgac (SEQ ID NO:1), which encodes a putative ATP-binding protein (SEQ ID NO:2) conferring resistance to A compounds, were carried out.

In particular, this invention provides a purified polynucleotide comprising the nucleotide sequence of SEQ ID NO: 1.

This invention additionally provides a purified peptide comprising an amino acid sequence of SEQ ID NO:2.

Furthermore, this invention provides a composition comprising a polynucleotide sequence encoding a molecule containing ATP binding motifs that confers resistance to Staphylococci and particularly to S. aureus, and wherein the polynucleotide sequence corresponds to a vgaC nucleotide sequence represented by SEQ ID NO:1 or a sequence having at least 85% homology with vgaC complete nucleotide sequence, or to a polynucleotide hybridizing with SEQ ID NO:1 under stringent conditions, or to a fragment containing between 20 and 30 contiguous nucleotides of SEQ ID NO:1, or wherein the polynucleotide sequence encodes a polypeptide having at least 60% homology with the complete SEQ ID NO:2.

Additionally, the invention includes a purified polynucleotide that hybridizes specifically under stringent conditions with a polynucleotide of SEQ ID NO: 1.

The invention further includes polynucleotide fragments comprising at least 10 nucleotides capable of hybridization under stringent conditions with any one of the nucleotide sequences enumerated above.

In another embodiment of the invention, a recombinant DNA sequence comprising at least one nucleotide sequence enumerated above and under the control of regulatory elements that regulate the expression of resistance to antibiotics of the streptogramin family in a defined host is provided.

Furthermore, the invention includes a recombinant vector comprising the recombinant DNA sequence noted above.

The invention also includes a recombinant host cell comprising a polynucleotide sequence enumerated above or the recombinant vector defined above.

In still a further embodiment of the invention, a method of detecting bacterial strains that contain the polynucleotide sequences set forth above is provided.

Additionally, the invention includes kits for the detection of the presence of bacterial strains that contain the polynucleotide sequences set forth above.

The invention also contemplates antibodies recognizing peptide fragments or polypeptides encoded by the polynucleotide sequences enumerated above.

Still further, the invention provides for a screening method for active antibiotics and/or molecules for the treatment of infections due to Gram-positive bacteria, particularly staphylococci, based on the detection of activity of these antibiotics and/or molecules on bacteria having the resistance phenotype to streptogramins.

It is to be understood that both the foregoing general description and the following detailed description are exemplary and explanatory only and are not restrictive of the invention, as claimed.

### BRIEF DESCRIPTION OF THE DRAWINGS

This invention will be described in greater detail with reference to the drawings in which:

FIG. 1 is a restriction map of the chromosomal region of BM3327 carrying vgaC (SEQ ID NO:1). The primers described in the section Materials and Methods and the sizes of the amplicons are indicated below the map. The nucleotide sequence of the vgaC (SEQ ID NO:1) gene and the flanking regions (190 nt upstream and 305 nt downstream) have been submitted to GenBank under the accession No. AF186237. Abbreviations: E, EcoRI; H, HindIII.

FIG. 2 contains amino acid sequences of Vga (6) (SEQ ID NO:3), VgaC (this invention; SEQ ID NO:2) and VgaB (3) (SEQ ID NO:4). The amino acids common to the three proteins are indicated by asterisks. The ATP-binding motifs described by Walker et al. (31) and the SGG signature sequences highly conserved in all investigated ATP-binding proteins (10, 20) are underlined. The relatively well conserved motifs in Vga and VgaC used as the basis of the degenerate primers A and B (Materials and Methods) are boxed.

FIG. 3 depicts PFGE of SmaI-digested total DNA from clinical S. aureus strains resistant to streptogramin A (A) SmaI macro-restriction patterns; (B) hybridization patterns with the vgaC-probe (pIP1799) at high stringency (65° C., 5×SSP buffer). Lanes 1, 8 and 9: bacteriophage lambda DNA concatermers (BioRad); lane 2: BM3385; lane 3: BM3250; lane 4: BM3252; lane 5: BM3249; lane 6: BM3327; lane 7: NCTC8325 used as standard; lane 10: BM3318.

FIG. 4A depicts the nucleotide sequence of vgaC (SEQ ID NO:1).

FIG. 4B depicts the amino acid sequence of VgaC (SEQ ID NO: 2).

### DETAILED DESCRIPTION OF THE INVENTION

It has now been determined that bacteria from the Staphylococcus genus carry a vgaC gene, which encodes a putative ATP-binding protein that confers resistance to streptogramin A and structurally similar compounds. The A gene, which has been designated vgaC (1575 bp), encoding a putative ABC protein conferring resistance to streptogramin A and related antibiotics was cloned from the chromosome of a S. aureus clinical isolate and sequenced. Despite its similarity with the vga gene, vgaC is distinct. This invention provides the vgaC gene and polynucleotide fragments thereof, the protein (VGAC) encoded by the gene, polypeptides derived from the protein, and their use in diagnostic and therapeutic applications.

In a specific embodiment of the present invention, the purified polynucleotides useful for detecting Staphylococcal strains can be used in combination in order to detect bacteria belonging to Gram positive bacteria in a biological sample. Thus, the present invention also provides detection methods and kits comprising combinations of the purified polynucleotides according to the invention. The purified oligonucleotides of the invention are also useful as primers for use in amplification reactions or as nucleic acid probes.

By “polynucleotides” according to the invention is meant the sequence referred to as SEQ ID NO:1, and the complementary sequences and/or the sequences of polynucleotides, which hybridize to the referred sequences in high stringent conditions and which are used for detecting staphylococcal strains carrying a gene encoding resistance to streptogramin A.

Thus, the polynucleotides of SEQ ID NO:1 and its fragments can be used to select nucleotide primers notably for an amplification reaction, such as the amplification reactions further described.

In a specific embodiment, the purified polynucleotides according to the present invention encompass polynucleotides having at least 85% homology in their nucleic acid sequences with polynucleotides of SEQ ID NO:1. By percentage of nucleotide homology according to the present invention is intended a percentage of identity between the corresponding bases of two homologous polynucleotides, this percentage of identity being purely statistical and the differences between two homologous polynucleotides being located at random and on the whole length of said polynucleotides.

The oligonucleotides according to the present invention hybridize specifically with a DNA or RNA molecule comprising all or part of one polynucleotide among SEQ ID NO:1 under stringent conditions. As an illustrative embodiment, the stringent hybridization conditions used in order to specifically detect a polynucleotide according to the present invention are advantageously the following:

Prehybridization and hybridization are performed at 68° C. in a mixture containing:

5×SSPE (1×SPE is 0.18 M NaCl, 10 mM NaH₂PO₄

5×Denhardt's solution

0.5% (w/v) sodium dodecyl sulfate (SDS); and

100 μg ml⁻¹ salmon sperm DNA

The washings are performed as follows:

Two washings at laboratory temperature for 10 min. in the presence of 2×SSPE and 0.1% SDS;

One washing at 68° C. for 15 min. in the presence of 1×SSPE, 0.1% SDS; and

One washing at 68° C. for 15 min. in the presence of 0.1×SSPE and 0.1% SDS.

The non-labeled polynucleotides or oligonucleotides of the invention can be directly used as probes. Nevertheless, the polynucleotides or oligonucleotides are generally labeled with a radioactive element (³²p ³⁵S, ³H ¹²⁵I) or by a non-isotopic molecule (for example, biotin, acetylaminofluorene, digoxigenin, 5-bromodesoxyuridin, fluorescein) in order to generate probes that are useful for numerous applications. Examples of non-radioactive labeling of nucleic acid fragments are described in the French Patent No. FR 78 10975 or by Urdea et al. or Sanchez-Pescador et al. 1988.

Other labeling techniques can also be used, such as those described in the French patents 2 422 956 and 2 518 755. The hybridization step may be performed in different ways (Matthews et al. 1988). A general method comprises immobilizing the nucleic acid that has been extracted from the biological sample on a substrate (nitrocellulose, nylon, polystyrene) and then incubating, in defined conditions, the target nucleic acid with the probe. Subsequent to the hybridization step, the excess amount of the specific probe is discarded, and the hybrid molecules formed are detected by an appropriate method (radioactivity, fluorescence, or enzyme activity measurement).

The oligonucleotide fragments according to the present invention can be prepared by cleavage of the polynucleotides of SEQ ID NO:1 by restriction enzymes, as described in Sambrook et al. in 1989. Another appropriate preparation process of the nucleic acids of the invention containing at most 200 nucleotides (or 200 bp if these molecules are double-stranded) comprises the following steps:

synthesizing DNA using the automated method of beta-cyanethylphosphoramidite;

cloning the thus obtained nucleic acids in an appropriate vector; and

purifying the nucleic acid by hybridizing to an appropriate probe according to the present invention.

A chemical method for producing the nucleic acids according to the invention, which have a length of more than 200 nucleotides (or 200 bp if these molecules are double-stranded), comprises the following steps:

Assembling the chemically synthesized oligonucleotides having different restriction sites at each end;

cloning the thus obtained nucleic acids in an appropriate vector; and

purifying the nucleic acid by hybridizing to an appropriate probe according to the present invention.

The oligonucleotides according to the present invention can also be used in a detection device comprising a matrix library of probes immobilized on a substrate, the sequence of each probe of a given length being localized in a shift of one or several bases, one from the other, each probe of the matrix library thus being complementary to a distinct sequence of the target nucleic acid. Optionally, the substrate of the matrix can be a material able to act as an electron donor, the detection of the matrix positions in which hybridization has occurred being subsequently determined by an electronic device. Such matrix libraries of probes and methods of specific detection of a target nucleic acid are described in the European patent application No. 0 713 016, or PCT Application No. WO 95 33846, or also PCT Application No. WO 95 11995 (Affymax Technologies), PCT Application No. WO 97 02357 (Affymetrix Inc.), and also in U.S. Pat. No. 5,202,231 (Drmanac), said patents and patent applications being herein incorporated by reference.

The present invention also pertains to a family of recombinant plasmids containing at least a nucleic acid according to the invention. According to an advantageous embodiment, a recombinant plasmid comprises a polynucleotide of SEQ ID NO: 1 or one nucleic fragment thereof, including, for example, the plasmid pIP1810, which has been deposited at the C.N.C.M. under Accession No. 1-2397.

The present invention is also directed to the full length coding sequence of the vgaC gene from Staphylococci (SEQ ID NO:1) that is available using the purified polynucleotides according to the present invention, as well as to the polypeptide encoded by these full length coding sequences. In a specific embodiment of the present invention, the full length coding sequence of the vgaC gene is isolated from a plasmid or cosmid library of the genome of Staphylococci that has been screened with the oligonucleotides according to the present invention. The selected positive plasmid or cosmid clones hybridizing with the oligonucleotides of the invention are then sequenced in order to characterize the corresponding full length coding sequence, and the DNA insert of interest is then cloned in an expression vector in order to produce the corresponding ATP binding motif conferring resistance to streptogramin A and related compounds.

A suitable vector for the expression in bacteria and in particular in E. coli, is the pQE-30 vector (QIAexpress) that allows the production of a recombinant protein containing a 6xHis affinity tag. The 6xHis tag is placed at the C-terminus or the N-terminus of the recombinant polypeptide ATP binding motif conferring resistance to streptogramin A and related compounds, which allows a subsequent efficient purification of the recombinant polypeptide ATP binding motif conferring resistance to streptogramin A and related compounds by passage onto a nickel or copper affinity chromatography column. The nickel chromatography column can contain the Ni-NTA resin (Porath et al. 1975).

The polypeptides according to the invention can also be prepared by conventional methods of chemical synthesis, either in a homogenous solution or in solid phase. As an illustrative embodiment of such chemical polypeptide synthesis techniques the homogenous solution technique described by Houbenweyl in 1974 may be cited.

The polypeptides according to the invention can be characterized by binding onto an immunoaffinity chromatography column on which polyclonal or monoclonal antibodies directed to a polypeptide outside the ATP binding motif conferring resistance to streptogramin A, and related compounds, have previously been immobilized.

Another object of the present invention comprises a polypeptide produced by the genetic engineering techniques or a polypeptide synthesized chemically as above described.

The polypeptide ATP binding motif conferring resistance to streptogramin A and related compounds according to the present invention is useful for the preparation of polyclonal or monoclonal antibodies that recognize the polypeptides or fragments thereof. The monoclonal antibodies can be prepared from hybridomas according to the technique described by Kohler and Milstein in 1975. The polyclonal antibodies can be prepared by immunization of a mammal, especially a mouse or a rabbit, with a polypeptide according to the invention that is combined with an adjuvant, and then by purifying specific antibodies contained in the serum of the immunized animal on a affinity chromatography column on which has previously been immobilized the polypeptide that has been used as the antigen.

Consequently, the invention is also directed to a method for detecting specifically the presence of a polypeptide according to the invention in a biological sample. The method comprises:

a) bringing into contact the biological sample with an antibody according to the invention; and

b) detecting antigen-antibody complex formed.

Also part of the invention is a diagnostic kit for in vitro detecting the presence of a polypeptide according to the present invention in a biological sample. The kit comprises:

a polyclonal or monoclonal antibody as described above, optionally labeled; and

a reagent allowing the detection of the antigen-antibody complexes formed, wherein the reagent carries optionally a label, or being able to be recognized itself by a labeled reagent, more particularly in the case when the above-mentioned monoclonal or polyclonal antibody is not labeled by itself.

Indeed, the monoclonal or polyclonal antibodies according to the present invention are useful as detection means in order to identify or characterize a Staphylococcal strain carrying genes encoding resistance to streptogramin A.

The invention also pertains to:

A purified polypeptide or a peptide fragment having at least 10 contiguous amino acids, which is recognized by antibodies directed against a polynucleotide sequence conferring resistance to streptogramin and related compounds, corresponding to a polynucleotide sequence according to the invention;

a polynucleotide comprising the full length coding sequence of a Staphylococcus streptogramin A resistant gene containing a polynucleotide sequence according to the invention;

a purified polypeptide having a molecular weight of approximately 58.2 kDa as determined by SDS-PAGE, which is encoded by a polynucleotide of the invention;

a monoclonal or polyclonal antibody directed against a polypeptide or a peptide fragment encoded by the polynucleotide sequences according to the invention;

a method of detecting the presence of bacterium harboring the polynucleotide sequences according to the invention in a biological sample comprising:

a) contacting bacterial DNA of the biological sample with a primer or a probe according to the invention, which hybridizes with a nucleotide sequence encoding resistance to streptogramins,

b) amplifying the nucleotide sequence using the primer or said probe, and

c) detecting the hybridized complex formed between the primer or probe with the DNA,

a kit for detecting the presence of bacterium having resistance to streptogramin A and harboring the polynucleotide sequences according to the invention in a biological sample, the kit comprising:

a) a polynucleotide probe according to the invention, and

b) reagents necessary to perform a nucleic acid hybridization reaction;

a kit for detecting the presence of bacterium having resistance to streptogramin A and harboring the polynucleotide sequences according to the invention in a biological sample, the kit comprising:

a) a polynucleotide probe according to the invention, and

b) reagents necessary to perform a nucleic acid hybridization reaction;

a method of screening active antibiotics for the treatment of the infections due to Gram-positive bacteria, comprising the steps of:

a) bringing into contact a Gram-positive bacteria having a resistance to streptogramin A and related compounds and containing the polynucleotide sequences according to the invention with the antibiotic, and

b) measuring an activity of the antibiotic on the bacteria having a resistance to streptogramins and related compounds;

a method of screening for active synthetic molecules capable of penetrating into a bacteria of the family of Gram positive bacteria, wherein the inhibiting activity of these molecules is tested on at least a polypeptide encoded by the polynucleotide sequences according to the invention comprising the steps of:

a) contacting a sample of the active molecules with the bacteria,

b) testing the capacity of the active molecules to penetrate into the bacteria and the capacity of inhibiting a bacterial culture at various concentration of the molecules, and

c) choosing the active molecule that provides an inhibitory effect of at least 80% on the bacterial culture compared to an untreated culture;

an in vitro method of screening for active molecules capable of inhibiting a polypeptide encoded by the polynucleotide sequences according to the invention, wherein the inhibiting activity of these molecules is tested on at least the polypeptide, the method comprising the steps of:

a) extracting a purified polypeptide according to the invention,

b) contacting the active molecules with the purified polypeptide,

c) testing the capacity of the active molecules, at various concentrations, to inhibit the activity of the purified polypeptide, and

d) choosing the active molecule that provides an inhibitory effect of at least 80% on the activity of the purified polypeptide;

a method of detecting the presence of bacterium harboring the polynucleotide sequences according to the invention in a biological sample, the method comprising the steps of:

a) contacting the sample with an antibody according to the invention that recognizes a polypeptide encoded by the polynucleotide sequences, and

b) detecting the complex; and

a diagnostic kit for in vitro detecting the presence of bacterium harboring the polynucleotide sequences according to the invention in a biological sample, the kit comprising:

a) a predetermined quantity of monoclonal or polyclonal antibodies according to the invention,

b) reagents necessary to perform an immunological reaction between the antibodies and a polypeptide encoded by said polynucleotide sequences, and

c) reagents necessary for detecting the complex between the antibodies and the polypeptide encoded by the polynucleotide sequences.

The inhibiting activity of the molecules can be readily evaluated by one skilled in the art. For example, the inhibiting activity can be tested by antibiogram with pristinamycin 2A, 20 μg per disk, in a manner analogous to the technique used for vgaA and vgaB genes for determination of CMI (inhibitor concentration), as described previously (2, 17).

By “active molecule” according to the invention is meant a molecule capable of inhibiting the activity of the purified polypeptide as defined in the present invention or capable of inhibiting the bacterial culture of Staphylococcal strains.

This invention will be described in greater detail in the following Examples.

### Materials and Methods

Bacterial strains and plasmids. The relevant characteristics of the strains and plasmids used are reported in Table 1.

TABLE 1
Relevant characteristics of the strains and plasmids used in this study.

| | Characteristics | | References |
|---|---|---|---|
| Strains | | | |
| BM3093 | S. aureus transductant: BM225 (pIP680: vga, | Fa Nv Rf SgB SgA (64 mg.1⁻¹)⁽¹⁾ Pt (4 mg.1⁻¹)⁽²⁾ | 4 |
| | vat, vgb) | | |
| BM3249 | S. aureus clinical isolate | L SgA (64 mg.1⁻¹) Pt (1 mg.1⁻¹) Pc Mc Tc Mn Km Nm Tm Gm Sm Su Pf | 1 |
| | | Fm Cd As Hg Eb | |
| BM3250 | S. aureus clinical isolate | L SgA (64 mg.1⁻¹) Pt (I mg.1⁻¹) Pc Mc Tc Mn Km Tm Gm Sm Su Cm Cd | 2, 3 |
| | | As Hg Eb | |
| BM3252 | S. aureus clinical isolate | L SgA (64 mg.1⁻¹) Pt (1 mg.1⁻¹) Pc Mc Tc Mn Kra Nm Tm Gm Sm Su Cd | 2, 3 |
| | | As Hg Eb | |
| BM3318 | S. aureus clinical isolate | MLSc SgA (128 mg.1⁻¹)Pt(16 mg.1⁻¹) Pc Mc Tc Mn Km Nm Tm Gm Sm | 2, 3 |
| | | Sp Su Cd As Hg Eb Ba | |
| BM3327 | S. aureus clinical isolate | MLSc SgA (128 mg.1⁻¹) Pt (8 mg.1⁻¹) Pc Tc Mn Km Nm Tm Gm Sm Su | 1, 3 |
| | | Pf | |
| BM3385 | S. aureus clinical isolate (pIP 1156: vgaB, | L SgA (128 mg.1⁻¹) Pt (4 mg.1⁻¹) Pc Su Tp Rf Cd As Eb | 2, 3 |
| | vgaC, vatB) | | |
| ISP1127 | S. aureus recipient | Nv | 26 |
| RN4220 | S. aureus recipient | No drug resistance marker | 22 |
| BM21926 | S. aureus transformant: RN4220 (pIP 1810) | SgA (32 mg.1⁻¹) Km Nm Tm | This study |
| BM21927 | S. aureus transformant: RN4220 (pRB374) | Km Nm Tm | This study |
| TOPIOF' | E. coli recipient (InVitrogen ®) | No drug resistance marker | |
| XL2-BLUE | E. coli recipient (Stratagène ®) | No drug resistance marker | |

| Plasmids | | | |
|---|---|---|---|
| pUC18 | | Ap | |
| pCR ®2.1- | E. coli cloning vector (InVitrogen ®) | Ap | |
| TOPO | | | |
| pRB374 | Shuttle vector with vegII promoter | Ap Km Nm Tm in E. coli; | 11 |
| | | Km Nm Tm in S. aureus | |
| pIP1652 | pUC18 + 619 bp insert from within vat, Accession n°: L07778: nt 269-nt 887 | Ap | 8 |
| pIP1653 | pUC18 + 476 bp insert from within vga, Accession n°: M90056: nt 1199-nt 1674 | Ap | 6 |
| pIP1654 | pUC18 + 920 bp insert from within vgb, Accession n°: JO3313: nt 624-nt 1543 | Ap | 7 |
| pIP1692 | pUC18 + 603 bp insert from within vatB, Accession n°: L38809: nt 79-nt 681 | Ap | 2 |
| pIP1705 | pUC18 + 1040 bp insert from within vgaB, Accession n°: U82085: nt 352-nt 1391 | Ap | 3 |
| pIP1740 | pUC18 + 565 bp insert from within vatC, Accession n°: AF015628: nt 1310-nt 1874 | Ap | 5 |
| pIP1741 | pUC18 + 714 bp insert from within vgbB, Accession n°: AFO15628: nt 520-nt 1233 | Ap | 5 |
| pIP1795 | pCR ®2.1-TOPO + 594 bp insert from within satA, Accession n°: L 12033: nt 189-nt 782 | Ap | This study |
| pIP1799 | pCR ®2.1-TOPO + 580 bp insert from within vgaC, Accession n°: AF186237: nt 668-nt | Ap | This study |
| | 1247 | | |
| pIP1802 | pUC18 + 528 bp from within satG, Accession n°: AF 153312: nt 363-nt 890 | Ap | 19 |
| pIP1810 | pRB374 + 1811 bp insert including vgaC from BM3327, Accession n°: AFI86237: nt 50- | Ap Km Nm Tm in E coli; | This study |
| | nt 1860 | SgA Km Nm Tm in S. | |
| | | aureus | |
| Abbreviations: Ap, ampicillin; AS, sodium arsenate; Ba, cetyltrimethylammonium bromide; Cd, cadmium acetate; Cm, chloramphenicol; Eb, ethidium bromide; Fa, fusicid acid; Fm, fosfomycin; Gm, gentamicin; Hg, mercuric nitrate; Km, kanamycin; L, lincosamide; MLSc, constitutive resistance to macrolides-lincosamides-streptogramin B; Mn, minocycline; Nm, neomycin; Pc, β-lactam (penicillinase production); Pf, pefloxacin; Rf, rifampin; SgA, streptogramin A; Sm, streptomycin; | | | |
| # Sp, spectinomycin; Su, sulfonamide; Tc, tetracycline; Tm, tobramycin; Tp, trimethoprim | | | |

⁽¹⁾Pristinamycin IIA MIC
⁽²⁾Pristinamycin MIC

Two collections of strains resistant to SgA were screened for the presence of vgaC. One collection consisted of (i) 52 staphylococci belonging to five species (S. aureus, S. epideridis, S. haemolyticus, S. cohnii subsp. urealyticum, and S. simulans) (1) and including the clinical isolates described in Table 1, and (ii) the S. cohnii strain harboring pIP1714 which carries vat C and vgbB (5). The other collection consisted of 51 E. faecium strains isolated from faecal samples from poultry, pigs, farmers, and suburban residents in the Netherlands (19).

Media

Staphylococci were grown in Brain Heart Infusion (Difco Laboratories, Detroit, Mich.), and E. coli cells were grown in Luria broth (10 g.1⁻¹ of tryptone, 5 g.⁻¹ of yeast extract, and 10 g.1⁻¹ of NaCl) Solid media contained 15 g.1⁻¹ of agar. Bacteria were incubated at 37° C. and for liquid cultures with agitation. Susceptibility to antibiotics was tested on Mueller-Hinton agar (MHA) (Diagnostics Pasteur, Marne-la-Coquette, France).

Susceptibility to Antimicrobial Drugs

Susceptibility to antibiotics was determined by a disk diffusion assay (12) with commercially available antibiotic disks (Diagnostics Pasteur) and disks prepared in our laboratory as described previously (18). MICs of pristinamycin IIA and pristinamycin (Rhône-Poulenc Rorer) were determined with serial twofold dilutions of antibiotics in MHA, as described previously (2, 17).

DNA isolation and analysis. Total cellular DNA was isolated from staphylococcal strains and was purified using the QIAamp tissue kit from Qiagen. Plasmid DNA was extracted and purified from Escherichia coli using the QIAprep spin plasmid kit from Qiagen. Restriction endonucleases were obtained from Amersham or Pharmacia and were used according to the manufacturer's instructions. Native or digested DNA was analysed by electrophoresis through 0.7% agarose gels. DNA fragments of less than 500 bp, amplified by PCR, were separated by electrophoresis through 4% NuSieve GTG agarose gels (FMC BioProducts). SmaI digestion and pulsed-field gel electrophoresis (PFGE) were performed as described previously (15).

Cloning and DNA Sequencing

Fragments amplified by PCR were cloned using the TOPOT™ TA cloning kit (Invitrogen) following the manufacturer's instructions. DNA restriction fragments were inserted into E. coli vectors using the ligase of the Fast-Link™ ligation kit (Epicentre Technologies Corporation) and the recombinant plasmids were introduced by transformation into competent E. coli XL2-blue cells (Stratagene) following the manufacturer's instructions.

An applied Biosystems automated 373R DNA sequencer, and the protocol described by the manufacturer were used for sequencing. The amino acid sequence deduced from the nucleotide sequence was analysed with the GCG package and compared with those deduced from nucleotide sequences in the GenBank/EMBL Database, as described in further detail below.

Labeling of DNA Probes, Blotting and Hybridization

Plasmid DNA was labeled with [α-³²] dCTP (110 Tbq mmol-⁻¹) by the random priming technique using the Megaprime DNA labeling system (Amersham).

Hybond N+ membranes (Amersham) were used for blotting. DNA was transferred from agarose gels to the membranes by the capillary blotting method of Southern (28). Blotted DNA was denatured and fixed to the membranes according to the protocol described in the Hybond N+ membranes, user handbook.

Prehybridization and hybridization were done at various temperatures (65° C., 60° C., 55° C., 50° C., 45° C., 42° C.) in a mixture containing 5×SSPE (1×SPE is 0.18 M NaCl, 10 mM NaH₂PO₄), 5×Denhardt's solution, 0.5% (w/v) SDS and 100 μg.ml⁻¹ fish sperm DNA (DNA, MB grade; Boehringer). The membranes carrying DNA transferred from agarose gels were treated with 10 ng.ml⁻¹ radiolabeled DNA probe. Washing was started with two successive immersions in 2×SSPE, 0.1% SDS, at room temperature for 10 min, followed by one immersion in 1×SSPE, 0.1% SDS, at the hybridization temperature for 15 min, and finally by one immersion in 0.1×SSPE, 0.1% SDS, at the hybridization temperature for 10 min. The washed blots treated with the radiolabeled probe were exposed to Hyperfilm™ (Amersham) at −80° C.

PCR

DNA was amplified by PCR using the kit <<Ready-To-Go™ >> (Amersham Pharmacia Biotech) according to the manufacturer's instructions in a Crocodile III apparatus (Appligéne). The following primers were used:

| | |
|---|---|
| A: 5′-AAYTAYWCNAAYTAYRTNGARCARAARGA-3′ | (SEQ ID NO:3); |
| (nt 1386-nt 1414 in vga, accession n° M90056) | |
| B: 5′-NACRTTYTCNARNATNGAYTT-3′ | (SEQ ID NO:4); |
| (nt 1967-nt 1947 in vga, accession n° M90056) | |
| C: 5′-CTTCAATTGGGATCCTCAGCATAGG-3′ | (SEQ ID NO:5); |
| BamH1 | |
| (nt 40-nt 64 in vgaC, accession n° AF186237) | |
| D: 5′-GTTATGGTACCTTCTTGTTAGG-′3 | (SEQ ID NO:6); |
| KPn 1 | |
| (nt 1866-nt 1845 in vgaC, accession n° AF186237) | |
| E: 5′-CTCTTTGTACGA GTATATGC-3′ | (SEQ ID NO:7); |
| (nt 612-nt 631 in vgaC, accession n° AF186237) | |
| F: 5′-GTTTCTTA GTAGCTCGTTGAGC-3′ | (SEQ ID NO:8) |
| (nt 809-nt 788 in vgaC, accession n° AF186237) | |

PCR experiments with primers A and B were carried out at low stringency (initial cycle of 5 min at 95° C. followed by 35 cycles of 30 sec at 40° C., 30 sec at 72° C. and 30 sec at 95° C. with a final extension step of 4 min at 40° C. and 10 min at 72° C.) and those with primers C and D, and E and F at high stringency (initial cycle of 5 min at 95° C. and 2 min at 55° C. followed by 35 cycles of 1 min at 72° C., 30 sec at 95° C. and 1 min at 55° C. and a final extension step of 5 min at 72° C.).

### EXAMPLE 1

Filter Mating Experiments

Each of the four S. aureus isolates (BM3249, BM3250, BM3252 and BM3327: Table 1) resistant to PIIA (MICs : 64-128 mg. 1⁻¹) and related antibiotics was crossed on a membrane filter with the S. aureus recipient strain, ISP1127 (26). No transcipients were obtained by selection on pristinamycin IIA (frequency<10⁻¹⁰ transcipients/donor CFU).

### EXAMPLE 2

### Hybridization Experiments with vga- and vgaB-probes at Various Temperatures

It has already been reported that the four S. aureus clinical isolates (BM3249, BM3250, BM3252 and BM3327) did not carry any of the known streptogramin A resistance genes (1, 3, 19). No amplicon was observed in PCR experiments with the degenerate primers M and N designed to detect genes encoding acetyltransferases inactivating streptogramin A. Moreover, the total cellular DNA of these strains did not hybridize at high stringency (65° C.) with the vat, vatB, vatC, satA, satG, vga, or vgaB probes (Table 1).

In this invention, these probes were used in hybridization experiments at 42° C., corresponding to the lowest temperature at which no non-specific background was observed with the chromosomal DNA of the S. aureus strain, RN4220 (22), susceptible to all antibiotics. At 42° C., a single HindIII fragment of 0.57 kb hybridizing with the vga-probe was revealed in the DNA of each of the four S. aureus clinical isolates (results not shown). An hybridizing HindIII fragment of the same size was detected in the DNA of the S. aureus clinical isolate, BM3385, harboring pIP1156 (˜60 kb), which carries vgaB and vatB, but the genes vgaB and vatB are contiguous and are located in a 7 kb HindIII fragment. In the S. aureus tranductant, BM3093, harboring pIP680 (4), the vga probe hybridized at 42° C. with a HindIII fragment of 5.6 kb carrying vga, confirming the absence of non-specific hybridization at 42° C.

To evaluate the highest temperature at which the vga probe revealed the presence of a 0.57 kb hybridizing HindIII fragment in the four S. aureus clinical isolates, hybridization experiments were carried out with the vga probe at 65° C., 60° C., 55° C., 50° C., and 45° C. The vga probe hybridized with the 0.57 kb HindIII fragment at 45° C., 50° C., and 55° C., but not at 60° C. and 65° C. Therefore, these isolates were suspected to carry a gene related to, but divergent from, vga.

### EXAMPLE 3

PCR Experiments with Degenerate Primers, A and B, that Encode Conserved Motifs in vga and vgaB

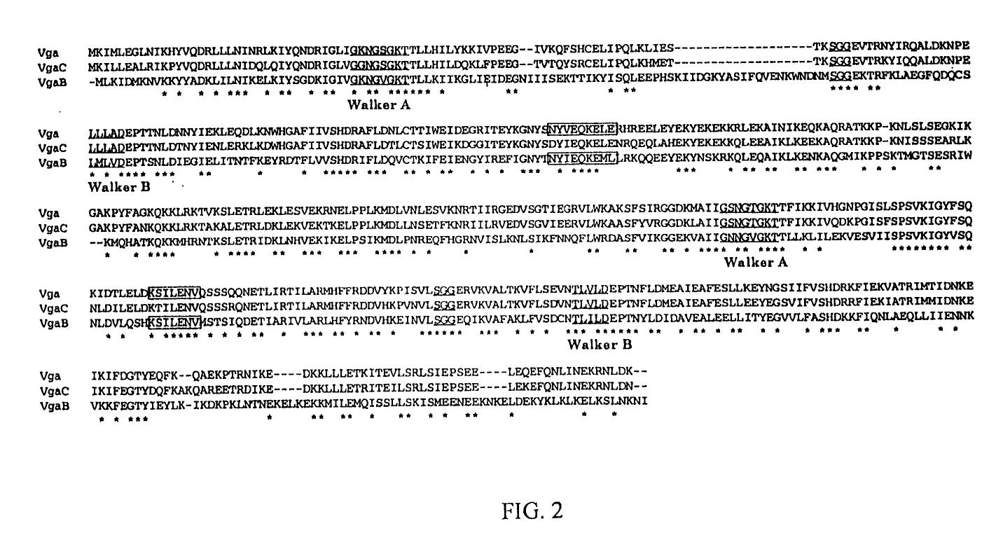

Conserved motifs in the peptide sequences of vga and vgaB (boxed in FIG. 2) were chosen away from the regions including the Walker motifs A and B, which are widespread in ABC proteins. The designed primers, A (coding-strand) and B (complementary-strand primer) were expected to amplify 582 bp and 579 bp DNA fragments from within the vga and vgaB genes, respectively.

The cellular DNA of BM3093 containing vga was primed with A and B in PCR experiments at low stringency (40° C.). A DNA fragment of the expected size (˜580 bp) was amplified. A DNA fragment of the same size was amplified from the cellular DNA of the five S. aureus clinical isolates, BM3249, BM3250, BM3252, BM3327, and BM3385. 514 nt of the 580 bp fragments amplified from BM3250 and BM3327 were sequenced. The two sequenced regions were identical and exhibited 80% and 61% similarity with vga and vgaB, respectively. The G+C contents of the amplicons (36.1%) were higher than those of vga (29%) or vgaB (27.2%).

The 580-bp amplicon from BM3250, which contained a single HindIII site and a single EcoRI site, was inserted in the linearised plasmid pCR®2.1-TOPO and the resulting recombinant plasmid was named pIP1799.

### EXAMPLE 4

### Hybridization Experiments Using pIP1799 as a Probe

Cellular DNA extracted from BM3249, BM3250, BM3252, BM3327, BM3093, and BM3385 was cleaved with HindIII and probed with pIP1799 at high stringency (65° C.) Nucleotide sequences hybridizing with the probe were detected in all the strains, except BM3093 (results not shown) Each hybridization pattern contained two or four HindIII fragments of which two (0.57 and 1.3 kb) were common to all the patterns; additional hybridizing fragments were detected in the patterns of BM3249 and BM3250 (3 kb), BM3252 (1.1 and 3 kb) and BM3385 (1.1 kb). These results suggested that the 580-bp insert of pIP1799, which hybridized neither with vga nor vgaB at high stringency, originated from a putative distinct gene, which will be named vgaC.

### EXAMPLE 5

### Cloning and Sequencing of the Putative vgaC Gene Carried by the Cellular DNA of BM3327

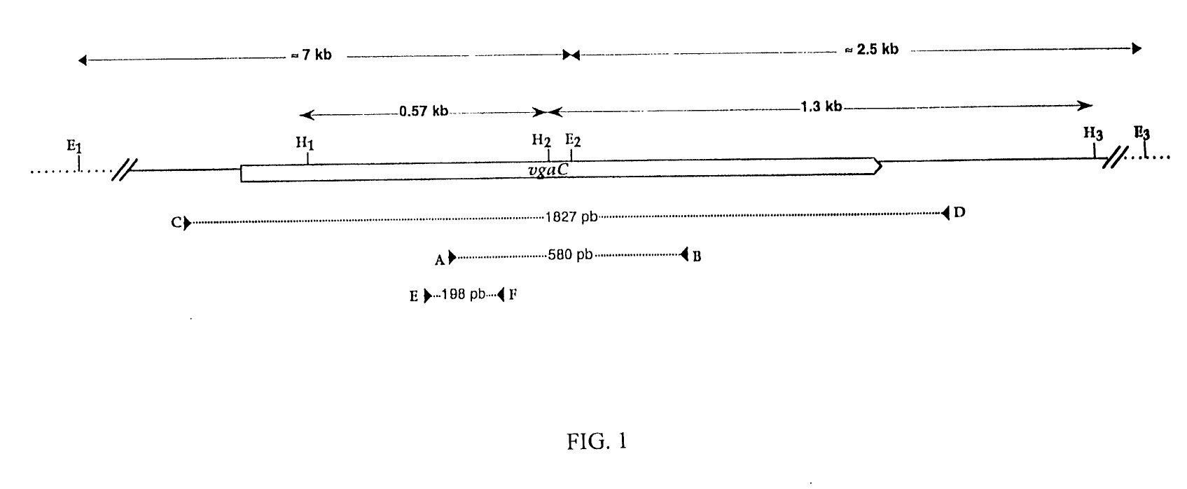

BM3327 was chosen because it carried only the two HindIII fragments of 0.57 and 1.3 kb hybridizing with pIP1799 and common to the hybridization patterns of the five S. aureus clinical isolates tested. These two fragments were expected to be contiguous since the sequence of the amplicon inserted in pIP1799 included a single HindIII site. Each of the fragments was inserted separately into the HindIII site of pUC18. The two recombinant plasmids were used to sequence the HindIII inserts of 0.57 and 1.3 kb. The sequence of each HindIII fragment was compared with that of the amplicon in pIP1799. Each HindIII insert contained part of the amplicon; thus they were contiguous and could be oriented (FIG. 1). An ORF of 1418 nt including the 0.57 kb HindIII insert (575 nt) and a 843-nt part of the 1.3 kb HindIII insert was found. Thus, the region of the genome upstream from the 0.57 kb HindIII fragment had to be obtained to sequence the N-terminal part of the putative gene.

Since the amplicon of pIP1799 carried a single EcoRI site, two EcoRI fragments hybridizing with pIP1799 were expected to be found in the cellular DNA of BM3327 and one of these EcoRI fragments would carry the putative N-terminal part of vgaC. Indeed, two EcoRI fragments of 2.5 and 7 kb were found in the hybridization pattern of BM3327 (results not shown). To identify the EcoRI fragment carrying the putative N-terminal-part of vgaC, a DNA fragment of 198 bp was amplified with oligonucleotides E and F (FIG. 1), using the cellular DNA of BM3327 as the template. The 198-bp fragment amplified with E and F did not exhibit any similarity with the other sequenced regions. The 198-bp amplicon used as a probe hybridized with the 7 kb EcoRI fragment of the cellular DNA of BM3327, but not with the 2.5 kb EcoRI fragment. Thus, the 7 kb EcoRI fragment was inserted into the EcoRI site of pUC18 and sequenced with a primer corresponding to a region within the 0.57 kb HindIII fragment (FIG. 1).

The first start codon (ATG) upstream from the HindIII site H₁ was preceded 8 nt upstream by 6 nt putative RBS. The DG of interaction of the most stable structure between this putative RBS and the 3′-terminus of the 16S rRNA (25), calculated according to Tinoco et al. (29), was −64.4 kJ.mol⁻¹.

Thus, the sequence registered in the GenBank EMBL-data Library under accession n^{o}AF186237, contains a 1575 bp gene, vgaC, delimited by the ATG codon at nt 191-193 and the TGA-stop codon at nt 1763-1765. This gene encodes a 524 aa putative protein of 58216 Da. The putative vgaC gene exhibited 83.2% nt identity with vga and 57.4% nt identity with the vgaB gene. The divergences between vga and vgaC are scattered along the whole genes.

The G+C content of vgaC is 35.6%. This value is higher than those of vga (29%) and vgaB (27.2%), but similar to those of the staphylococcal genome (32-36%).

### EXAMPLE 6

### Analysis of the Drug-resistance Pattern Conferred by VgaC

A DNA fragment of 1827-bp including vgaC was amplified with primers C and D (FIG. 1) from the cellular DNA of BM3327. This amplicon was cleaved with BamHl and KpnI and inserted between the BamHl and KpnI sites of the shuttle vector, pRB374 (11), downstream from the vegII promoter carried by this vector. The resulting plasmid, pIP1810, was introduced by electroporation into the S. aureus recipient RN4220. It conferred resistance to pristinamycin IIA (MICs: 32 mg.1⁻¹ for RN4220 [pIP18101, and 2 mg.1⁻¹ for RN4220 [pRB374]).

The plasmid pIP1810 was deposited on Jan. 21, 2000, at the Collection Nationale de Cultures de Microorganismes (C.N.C.M.), 25, Rue de Docteur Roux, F-75724, Paris Cedex 15, France, under Accession No. I-2379.

### EXAMPLE 7

### Analysis of the VgaC Amino Acid Sequence

The predicted translation product of the vgaC gene, VgaC, has a calculated pI of 7.27. The hydropathy plot of the VgaC sequence according to the algorithm of Kyte and Doolittle (23) indicates the protein to be hydrophilic. It contained no sequence similar to known signal sequences of secreted proteins (30).

The aa sequence of VgaC was compared to the sequences available in databases (GenBank, release 114; EMBL, Release 60). Program GAP, “GCG” (Genetic Computer Group) from Program Manual (UNIX), Wisconsin Sequence Analysis Package™, Algorithm of Needleman and Wunsch. The parameters are chosen as follows:

a) for amino acid comparisons:

gap penalty: 5

gap extension penalty: 0.30

length: the sequence to be compared in SEQ ID NO:2 having 524 amino acids.

b) for nucleotide comparisons:

gap penalty: 50

gap extension penalty: 3.

Significant similarities to the ATP-binding domains of numerous ABC proteins were found. The proteins giving the best matches were Vga (81.2% identical aa) and VgaB, (57.4% identical aa). The aa sequence alignment of Vga, VgaB and VgaC is shown in FIG. 2. Each of the three proteins contain two ATP-binding domains including, each, the two ATP-binding motifs described by Walker et al. (31) and a highly conserved SGG signature sequence found between the two ATP-binding motifs of all investigated ATP-binding proteins (10, 20).

### EXAMPLE 8

### Distribution and Location of the vgaC Gene in Two Collections of Isolates Resistant to A Compounds

One of these collections consisted of 53 staphylococcal isolates and the other of 51 E. faecium isolates from faecal samples from farmers and animals (Material and Methods). The total cellular DNA of these isolates was subjected to agarose gel electrophoresis and probed with pIP1799 at high stringency (65° C.). Sequences hybridizing with this probe were found in the 20 S. aureus isolates carrying vgaB and vatB (1) including BM3318 and BM3385 (Table 1), in one S. epidermidis isolates carrying vga only (1), and in the four S. aureus isolates, i.e. BM3249, BM3250, BM3252, and BM3327 (Table 1). The hybridizing sequences co-migrated with the chromosomal fragment DNA in all isolates and in 15 of them an additional signal was detected in extrachromosomal DNA bands (≧40 kb) (results not shown).

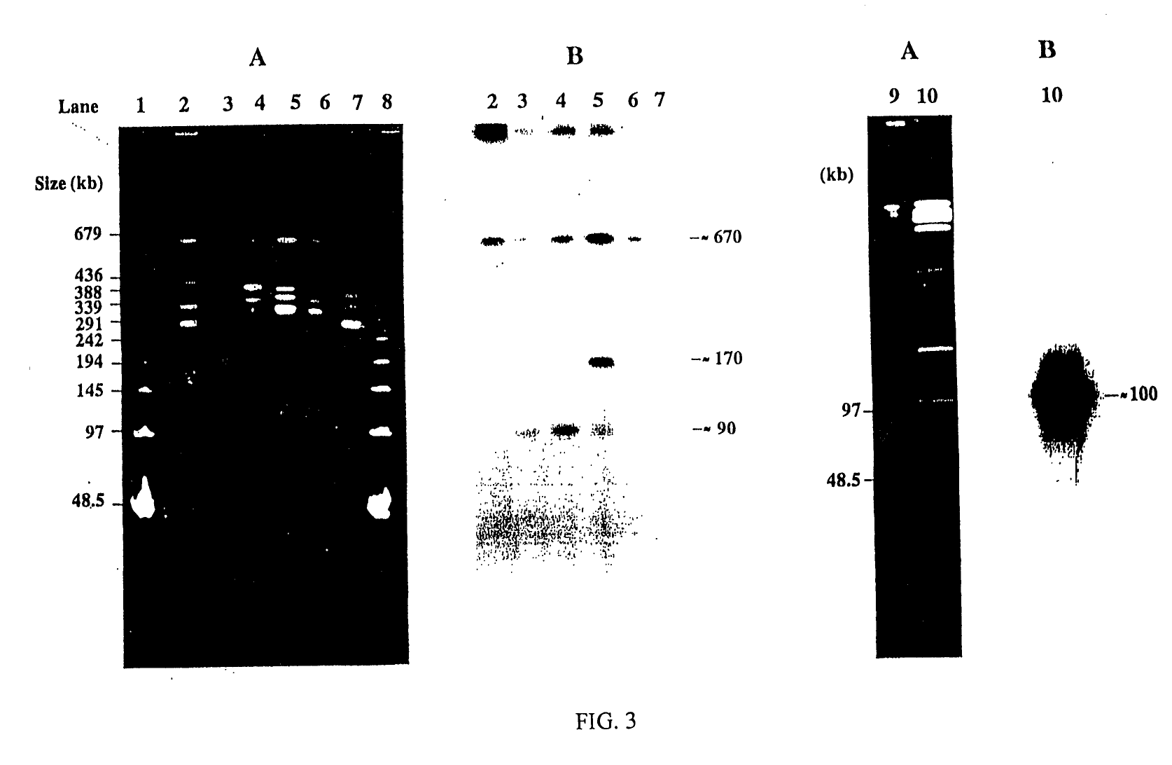

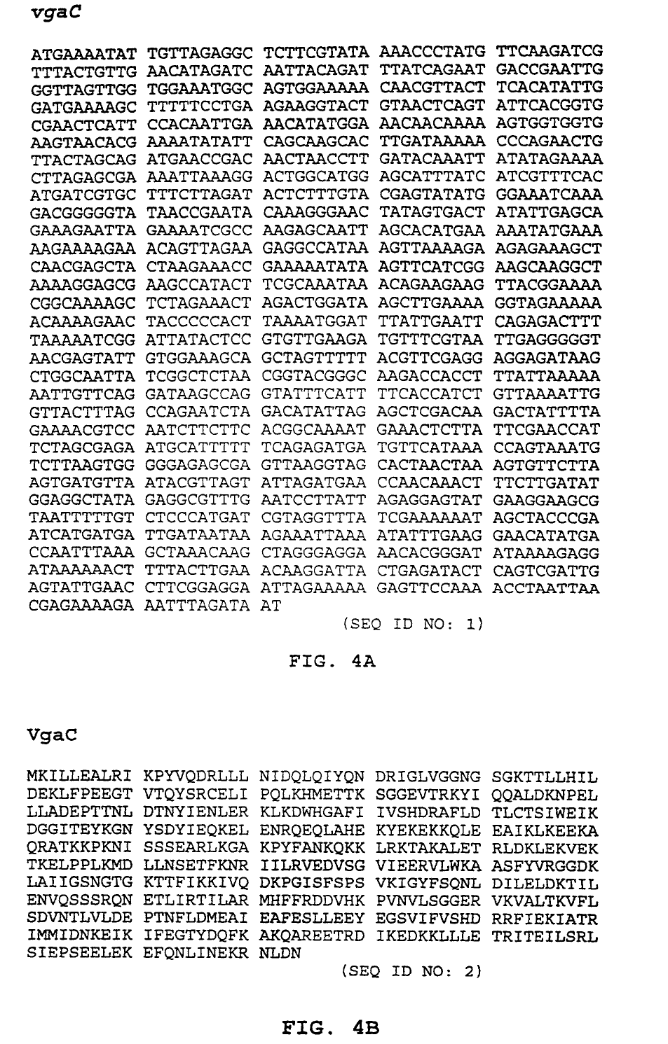

The cellular DNA of six S. aureus clinical isolates hybridizing with pIP1799 was digested with SmaI and probed with this plasmid at 65° C. (FIG. 3). A 670 kb SmaI hybridizing fragment was revealed in BM3385, BM3250, BM3252, BM3249, and BM3327 (FIG. 3B, lanes 2-6). An additional SmaI fragment of 90 kb was detected in BM3250 and BM3252 (FIG. 3, lanes 3 and 4), and two additional SmaI fragments of 90 kb and 170 kb were present in the pattern of BM3249 (lane 5).

In BM3318, the hybridizing SmaI fragment was 100 kb (FIG. 3B, lane 10). The same band also hybridized with vatB and vgaB probes (results not shown) suggesting that this 100 kb fragment originated from a plasmid carrying three SgA^{R} genes. In the lane of BM3385 (FIG. 3B, lane 2) pIP1799 hybridized with DNA, which did not migrate out of the well, and a similar signal was observed in hybridizations with vatB and vgaB probes (results not shown). BM3385 contains pIP1156, which has no SmaI site and carries functional vatB and vgaB genes. Presumably this plasmid also contains nucleotide sequences hybridizing with pIP1799.

### EXAMPLE 9

### Screening for Complete Copies of vgaC

To check whether BM3249, BM3250, BM3252, BM3318, BM3327, and BM3385, which hybridize with pIP1799, carry a complete copy of vgaC, PCR experiments were carried out using primers C and D (FIG. 1). For each of the tested strains, the size of the amplified fragments was the same as that of the BM3327 amplicon, suggesting that each strain carries at least one copy of vgaC and the adjacent regions (152 nt upstream and 102 nt downstream from the BM3327 vgaC gene).

Despite the great similarity with the vga gene (82%), the vgaC gene conferring resistance to streptogramin A can be considered to be distinct because of the absence of hybridization at high stringency (60° C.). This result may be attributed to the differences in the G+C contents of the two genes (29% for vga and 35% for vgaC) and the distribution of the divergences along the whole nucleotide sequence.

There are at least two reasons why the G+C content of vgaC is closer to that of staphylococcal genome (32-36%) than the G+C content of vga or vgaB (27-29%): horizontal transfer from different species whose genomes have different G+C contents; or their acquisition, at different times, from the same species, which has a lower G+C content. In the latter case, vgaC would have been introduced into staphylococci earlier than vga or vgaB, such that it has evolved towards G+C content similar to that of staphylococcal genome.

Previously described SgA^{R} genes are always found as single copies on plasmids. In contrast, the vgaC gene is present in multiple copies in some clinical isolates and is found either on the chromosome (BM3249, BM3250, BM3252) or both on the chromosome and a plasmid (BM3385). The multiplicity of copies does not correlate with higher levels of resistance to streptogramin A, raising the question of whether all copies carried by the same strain are functional. Cloning the various copies of vgaC would provide an answer to this question.

Nucleotide sequences hybridizing specifically with vgaC were detected in all the staphylococcal isolates harboring plasmids which carry vgaB vatB. There is no explanation for this finding. Surprisingly, none of the staphylococcal genes encoding ABC proteins which confer resistance to streptogramin A (vga, vgaB and vgaC), were found in E. faecium strains resistant to this antibiotic. In these strains, satA and satG encoding acetyltransferases inactivating streptogramin A, are widespread (19, 21, 32). However, satA and satG are never detected in staphylococci (19). The only gene conferring resistance to streptogramin A common to both genera is vat found close to vgb and in the same relative positions in the enterococcal and staphylococcal plasmids (19).

All attempts to detect a transposon carrying vga failed in seven staphylococcal plasmids (24-45 kb) carrying vga, vat, and vgb (4) and in two S. epidermidis plasmids (7.5-14.4 kb) carrying vga (9, 24). In contrast with vga, which is disseminated by plasmids, vgaC may have been disseminated by a transposon since, depending on the strain, it is carried by a vatB-vgaB plasmid (BM3318), by a vatB-vgaB plasmid and by the chromosome (BM3385) or by the chromosome in which multiple copies may be present. Sequencing of the regions adjacent to vgaC is required to determine whether or not it is part of a transposon.

In summary, the new gene, which has been designated vgaC (1575 bp), encoding an ABC protein conferring resistance to streptogramin A and related antibiotics was cloned from the chromosome of a S. aureus clinical isolate and sequenced. Despite its similarity with the vga gene (82%), vgaC can be considered to be distinct because of its higher content in G+C (35% for vgaC and 29% for vga) and because of the absence of hybridization at high stringency (60° C.), cross hybridization between vga and vgaC being detected at 55° C. and lower temperatures. Unlike other vga genes, vgaC is present in multiple copies in some clinical isolates. These copies are chromosomal in some isolates and both on the chromosome and on plasmids in others. Surprisingly, nucleotide sequences hybridizing at 65° C. with vgaC were found in all the staphylococcal strains harboring plasmids that carry two other genes encoding resistance to streptogramin A, vgaB and vatB.

### REFERENCES

The following references have been cited in this application. The entire disclosure of each reference is relied upon and incorporated by reference herein.

1. Allignet, J., S. Aubert, A. Morvan, and N. El Solh. 1996. Distribution of the genes encoding resistance to streptogramin A and related compounds among the staphylococcic resistant to these antibiotics. Antimicrobial Agents and Chemotherapy 40:2523-2528.

2. Allignet, J., and N. El Solh. 1995. Diversity among the Gram-positive acetyltransferases inactivating streptogramin A and structurally related compounds, and characterization of new staphylococcal determinant, vatB. Antimicrobial Agents and Chemotherapy 39:2027-2036.

3. Allignet, J., and N. El Solh. 1997. Characterization of a new staphylococcal gene, vgaB, encoding a putative ABC transporter conferring resistance to streptogramin A and related compounds. Gene 202:133-138.

4. Allignet, J., and N. El Solh. 1999. Comparative analysis of staphylococcal plasmids carrying three streptogramin-resistance genes vat vgb vga. Plasmid 42:134-138.

5. Allignet, J., N. Liassine, and N. El Solh. 1998. Characterization of a staphylococcal plasmid related to pUB110, pIP1714, and carrying two novel genes, vatC and vgbB, encoding resistance to streptogramins A and B and similar antibiotics. Antimicrob. Agents Chemother. 42:1794 1798.

6. Allignet, J., V. Loncle, and N. El Solh. 1992. Sequence of a staphylococcal plasmid gene, vga, encoding a putative ATP-binding protein involved in resistance to virginiamycin A-like antibiotics. Gene 117:45-51.

7. Alignet, J., V. Loncle, P. Mazodier, and N. El Solh. 1988. Nucleotide sequence of a staphylococcal plasmid gene, vgb, encoding a hydrolase inactivating the B components of viriginiamycin A-like antibiotics. Plasmid 20:271-275.

8. Allignet, J., V. Loncle, C. Simenel, M. Delepierre, and N. El Solh. 1993. Sequence of a staphylococcal genef vat, encoding an acetyltransferase inactivating the A-type compounds of virginiamycin-like antibiotics. Gene 130:91-98.

9. Aubert, S., K. G. H. Dyke, and N. El Solh. 1998. Analysis of two Staphylococcus epidermidis plasmids coding for resistance to streptogramin A. Plasmid 40:238-242.

10. Barrasa, M. I., J. A. Tercero, R. A. Lacalle, and A. Jimenez. 1995. The ardl gene from Streptomyces capreolus encodes a polypeptide of the ABC-transporters superfamily which confers resistance to the aminonucleotide antibiotic A201A. Eur. J. Biochem. 228:562-569.

11. Bruckner, R. 1992. A series of shuttle vectors for Bacillus subtilis and Escherichia coli. Gene 122:187-192.

12. Chabbert, Y. A. 1982. Sensibilité bactérienne aux antibiotiques, p. 204-212. In L. Le Minor and M. Veron (ed.), Bactériologie Médicale. Flammarion, Médecine Science, Paris.

13. Cocito, C. 1979. Antibiotics of the virginiamycin family, inhibitors which contain synergistic components. Microbiol. Rev. 43:145-198.

14. Cocito, C., M. Digambattista, E. Nyssen, and P. Vannuffel. 1997. Inhibition of protein synthesis by streptogramins and related antibiotics. J. Antimicrob. Chemother. 39 (Suppl. A) :7-13.

15. Derbise, A., K. G. H. Dyke, and N. El Solh. 1996. Characterization of a Staphylococcus aureus transposon Tn5405, located within Tn5404 and carrying the aminoglycoside resistance genes, aphA-3 and aadE. Plasmid 35:174-188.

16. El Solh, N., and J. Allignet. 1998. Staphylococcal resistance to streptogramins and related antibiotics. Drug Resist. Updates 1:169-175.

17. Ericson, H. M., and J. C. Sherris. 1971. Antibiotic susceptibility testing. Report of an international Collaborative Study. Acta Pathol. Microbiol. Scand. Suppl.217(Section B):11-90.

18. Galdbart, J. -0., A. Morvan, N. Desplaces, and N. El Solh. 1999. Phenotypic and genomic variation among Staphylococcus epidermidis strains infecting joint prostheses. J. Clin. Microbiol. 37:1306-1312.

19. Haroche, J., J. Allignet, S. Aubert, A. E. van den Bogaard, and N. El Solh. 1999. satG, conferring resistance to streptogramin A, is widely distributed in Enterococcus faecium strains but not in staphylococci. Antimicrob. Agents Chemother.:In press.

20. Hyde, S. C., P. Emsley, M. J. Hartshorn, M. M. Mimmack, U. Gileadi, S. R. Pearce, M. P. Gallagher, D. R. Gill, R. E. Hubbard, and C. F. Higgins. 1990. Structural model of ATP-binding proteins associated with cystic fibrosis, multidrug resistance and bacterial transport. Nature 346:362-365.

21. Jensen, L. B., A. M. Hammerum, F. M. Aarestrup, A. E. van den Bogaard, and E. E. Stobberingh. 1998. Occurrence of satA and vgb genes in streptogramin-resistant Enterococcus faecium isolates of animal and human origins in The Netherlands. Antimicrob. Agents Chemother. 42:3330-3331.

22. Kreiswirth, B. N., S. Lofdahl, M. J. Bethey, M. O'Reilly, P. M. Shlievert, M. S. Bergdoll, and R. P. Novick. 1983. The toxic shock exotoxin structural gene is not detectably transmitted by a prophage. Nature 306:709-712.

23. Kyte, J., and R. F. Doolittle. 1982. A simple method for displaying the hydropathic character of a protein. J. Mol. Biol. 157:105-132.

24. Loncle, V., A. Casetta, A. BuuHoi, and N. El Solh. 1993. Analysis of pristinamycin-resistant Staphylococcus epidermidis isolates responsible for an outbreak in a parisian hospital. Antimicrobial Agents and Chemotherapy 37:2159-2165.

25. Moran, C. P., Jr., N. Lang, S. F. J. LeGrice, G. Lee, M. Stephens, A. L. Sonenshein, J. Pero, and R. Losick. 1982. Nucleotide sequences that signal the initiation of transcription and translation in Bacillus subtilis. Mol. Gen. Genet. 186:339-346.

26. Pattee, P. A., and D. S. Neveln. 1975. Transformation analysis of three linkage groups in Staphylococcus aureus. J. Bacteriol. 124:201-211.

27. Rende-Fournier, R., R. Leclercq, M. Galimand, J. Duval, and P. Courvalin. 1993. Identification of the satA gene encoding a streptogramin A acetyltransferase in Enterococcus faecium BM4145. Antimicrobial Agents and Chemotherapy 37:2119-2125.

28. Southern, E. M. 1975. Detection of specific sequences among DNA fragments separated by gel electrophoresis. J. Mol. Biol. 98:503-517.

29. Tinoco, I., Jr., P. N. Borer, B. Dengler, M. D. Levine, 0. C. Uhlenbeck, D. M. Crothers, and J. Gralla. 1973. Improved estimation of secondary structure in ribonucleic acids. Nature New Biology 246 :40-41.

30. von Heijne, G. 1986. A new method for predicting signal sequence cleavage sites. Nucleic Acids Res. 14:4683-4690.

31. Walker, J. E., M. Saraste, M. J. Runswick, and N. J. Gay. 1982. Distantly related sequences in the a- and β-subunits of ATP synthase, myosin, kinases and other ATP-requiring enzymes and a common nucleotide binding fold. EMBO Journal (European Molecular Biology Organization) 1:945-951.

32. Werner, G., and W. Witte. 1999. Characterization of a new enterococcal gene, satg, encoding a putative acetyltransferase conferring resistance to streptogramin A compounds. Antimicrob. Agents Chemother. 7:1813-1814.

What is claimed is:

1. A purified nucleic acid molecule of SEQ ID NO: 1.

2. A purified nucleic acid molecule encoding a peptide of SEQ ID NO: 2.

3. A purified nucleic acid molecule that hybridizes to either strand of a denatured, double-stranded DNA comprising the nucleic acid molecule of any one of claims 1 or 2 under conditions of moderate stringency.

4. The purified nucleic acid molecule as claimed in claim 3, wherein said isolated nucleic acid molecule is derived by in vitro mutagenesis from SEQ ID NO: 1.

5. A purified nucleic acid molecule degenerate from SEQ ID NO: 1 as a result of the genetic code.

6. A purified nucleic acid molecule of SEQ ID NO: 1 without mutations.

7. A purified nucleic acid molecule, which encodes VGAC polypeptide, an allelic variant of VGAC polypeptide or a homolog of VGAC polypeptide.

8. A recombinant vector that directs the expression of a nucleic acid molecule selected from the group consisting of the purified nucleic acid molecules of claims 1, 2, 4, 5, 6, and 7.

9. A recombinant vector that directs the expression of a nucleic acid molecule of claim 3.

10. A recombinant vector that directs the expression of a nucleic acid molecule of claim 4.

11. A purified polypeptide encoded by a nucleic acid molecule selected from the group consisting of the purified nucleic acid molecules of claims 1, 2, 4, 5, 6, and 7.

12. A purified polypeptide according to claim 10 having a molecular weight of approximately 58.2 kDa as determined by SDS-PAGE.

13. A purified polypeptide according to claim 12 in post translationally modified form or not.

14. A purified polypeptide encoded by a nucleic acid molecule of claim 3.

15. A purified polypeptide according to claim 14 in post translationally modified form or not.

16. A purified polypeptide encoded by a nucleic acid molecule of claim 4.

17. A purified polypeptide according to claim 16 in post translationally modified form or not.

18. Purified antibodies that bind to a polypeptide of claim 11.

19. Purified antibodies according to claim 18, wherein the antibodies are monoclonal antibodies.

20. Purified antibodies that bind to a polypeptide of claim 14.

21. Purified antibodies according to claim 20, wherein the antibodies are monoclonal antibodies.

22. Purified antibodies that bind to a polypeptide of claim 16.

23. Purified antibodies according to claim 22, wherein the antibodies are monoclonal antibodies.

24. A host cell transfected or transduced with the vector of claim 8.

25. A method for the production of VGAC polypeptide comprising culturing a host cell of claim 24 under conditions promoting expression, and recovering the polypeptide from the host cell or the culture medium.

26. The method of claim 25, wherein the host cell is selected from the group consisting of bacterial cells, parasite cells and eukaryotic cells.

27. A host cell transfected or transduced with the vector of claims 8 to 10.

28. A method for the production of VGAC polypeptide comprising culturing a host cell of claim 27 under conditions promoting expression, and recovering the polypeptide from the host cell or the culture medium.

29. The method of claim 28, wherein the host cell is selected from the group consisting of bacterial cells, parasite cells and eukaryotic cells.

30. A host cell transfected or transduced with the vector of claim 10.

31. A method for the production of VGAC polypeptide comprising culturing a host cell of claim 30 under conditions promoting expression, and recovering the polypeptide from the host cell or the culture medium.

32. The method of claim 31, wherein the host cell is selected from the group consisting of bacterial cells, or parasitic cells or eukaryotic cells.

33. An immunological complex comprising a VGAC polypeptide and an antibody that specifically recognizes said polypeptide.

34. A method of detecting a microorganism in a biological sample that harbors a polynucleotide sequence according to claim 1, said method comprising the steps of:
(a) contacting microorganism DNA of the biological sample with a primer or a probe, which hybridizes with the polynucleotide sequence of claim 1;
(b) amplifying the nucleotide sequence using said primer or said probe; and
(c) detecting a hybridized complex formed between said primer or probe and the DNA.

35. A method of detecting a microorganism in a biological sample that harbors a polypeptide according to claims 11 to 17 or fragments or peptides thereof, which can be recognized by antibodies raised against VGAC, said method comprising the steps of:
(a) contacting the biological sample with antibodies according to claims 18 to 23; and
(b) detecting the resulting immunocomplex.

36. A kit for detecting a microorganism that harbors a polynucleotide sequence according to claim 1, said kit comprising:
(a) a polynucleotide probe that hybridizes with the polynucleotide sequence of claim 1; and
(b) reagents to perform a nucleic acid hybridization reaction.

37. A kit according to claim 35 comprising:
(a) purified antibodies according to claims 18 to 23, which react with the polypeptide as claimed in claims 10 to 16;
(b) standard reagents; and
(c) detection reagents.

38. A composition comprising at least one polypeptide encoded by vgaC.

39. A composition comprising at least one nucleotide sequence according to claim 1 that encodes resistance to streptogramins or induces streptogramin resistance in Gram-positive bacteria.

40. The composition according to claim 39, wherein said composition comprises at least a nucleotide sequence encoding a molecule containing ATP binding motifs conferring resistance to a streptogramin.

41. A composition comprising a polynucleotide sequence that encodes a molecule containing ATP binding motifs, which confer resistance to a streptogramin in Staphylococcus and wherein the polynucleotide sequence is selected from the group consisting of:
a) SEQ ID NO: 1;
b) a polynucleotide sequence having at least 70% of identity with SEQ ID NO: 1;
c) a polynucleotide sequence hybridizing with said SEQ ID NO: 1 under stringent conditions or to a fragment containing between 20 and 30 contiguous nucleotides of SEQ ID NO: 1;
d) a polynucleotide sequence that encodes a polypeptide having at least 60% homology with SEQ ID NO: 2; and
e) a polynucleotide sequence having at least 85% homology with SEQ ID NO: 1.

42. A composition of claim 41, wherein the polynucleotide sequence encoding a molecule containing ATP binding motifs confers resistance to a streptogramin in Staphylococcus aureus.

43. A polynucleotide fragment comprising at least 10 contiguous nucleotides that hybridizes under stringent conditions with a sequence according to claim 1 or the polynucleotide complementary fragment thereof.

44. A recombinant DNA molecule comprising at least one nucleotide sequence according to claim 1 under the control of regulatory elements that regulate the expression of resistance to antibiotics of the streptogramin family in a host.

45. A recombinant cell host comprising SEQ ID NO: 1, or the recombinant molecule of claim 44.

46. A purified polypeptide or a peptide fragment having at least 10 amino acids, which is recognized by antibodies directed against a peptide encoded by a polynucleotide sequence conferring resistance to a streptogramin corresponding to a polynucleotide sequence according to claim 1.

47. A kit for detecting a bacterium that is resistant to a streptogramin and harbors a polynucleotide sequence according to claim 2, said kit comprising:
a) a polynucleotide probe that hybridizes with the polynucleotide sequence of claim 2; and
b) reagents to perform a nucleic acid hybridization reaction.

48. A method of screening an active antibiotic for treating a Gram-positive bacterial infection, comprising the steps of:
a) contacting the antibiotic with Gram-positive bacteria that are resistant to a streptogramin and contain a polynucleotide sequence according to claim 1; and
b) determining the activity of the antibiotic on the bacteria.

49. A method of screening for active synthetic molecules capable of penetrating into a bacteria of the staphylococci family, wherein an inhibiting activity of the molecules is tested on at least a polypeptide encoded by a polynucleotide sequence according to claim 1, the method comprising the steps of:
a) contacting a sample of said active molecules with the bacteria;
b) testing the capacity of the active molecules to penetrate into the bacteria and the capacity of inhibiting a bacterial culture at various concentration of the molecules; and
c) choosing the active molecule that provides an inhibitory effect of at least 80% on the bacterial culture compared to an untreated culture.

50. An in vitro method of screening for active molecules capable of inhibiting a polypeptide encoded by a polynucleotide sequence according to claim 1, said method comprising the steps of:
a) contacting the active molecules with said polypeptide;
b) testing the capacity of the active molecules, at various concentrations, to inhibit the activity of the polypeptide; and
c) choosing the active molecule that provides an inhibitory effect of at least 80% on the activity of the said polypeptide.

51. A method of detecting a bacterium in a biological sample that harbors a polynucleotide sequence according to claim 2, said method comprising the steps of:
a) contacting said sample with an antibody according to claim 18 that recognizes a polypeptide encoded by said polynucleotide sequences; and
b) detecting a complex formed between the antibody and the polypeptide.

52. A diagnostic kit for in vitro detection of a bacterium harboring the polynucleotide sequences according to claim 2, said kit comprising:
a) a predetermined quantity of monoclonal or polyclonal antibodies according to claim 18;
b) reagents to perform an immunological reaction between the antibodies and a polypeptide encoded by said polynucleotide sequences; and
c) reagents for detecting a complex formed between the antibodies and the polypeptide encoded by said polynucleotide sequences.

53. A plasmid deposited at the C.N.C.M. under Accession No. 1-2379.
